(19) United States
(12) Patent Application Publication
(10) Pub. No.: US 2025/0388943 A1
(43) Pub. Date: Dec. 25, 2025

(54) SELF-ALIGNMENT-BASED PRECISE DNA OPTICAL SYNTHESIS THROUGH IMAGE INVERSION

(71) Applicant: KOREA UNIVERSITY RESEARCH AND BUSINESS FOUNDATION, Seoul (KR)

(72) Inventor: Hong Gu CHUN, Seoul (KR)

(73) Assignee: KOREA UNIVERSITY RESEARCH AND BUSINESS FOUNDATION, Seoul (KR)

(21) Appl. No.: 18/857,123

(22) PCT Filed: Apr. 13, 2023
(86) PCT No.: PCT/KR2023/004979

§ 371 Date: Aug. 11, 2025

(30) Foreign Application Priority Data
Apr. 13, 2022 (KR) 10-2022-0045903

(51) Int. Cl. C12P 19/34

(52) U.S. Cl. CPC C12P 19/34

(57) ABSTRACT

The present invention is a technology that can reduce errors occurring due to insufficient light in the DNA synthesis process, wherein DNA is deprotected and capped first in areas other than the desired DNA synthesis area and then deprotected and coupled in the desired DNA synthesis area, whereby unwanted DNA deprotection can be prevented and, as a result, DNA synthesis errors can be reduced.

### TECHNICAL FIELD

The present invention relates to a method for precision DNA optical synthesis, and more particularly, to a technology that can reduce DNA synthesis errors by preventing light from deprotecting DNA in an undesired region in a photochemical-based DNA synthesis process to reduce DNA synthesis errors.

### BACKGROUND ART

Due to the increase in personal social network service (SNS) media, mainly images and videos, data production is growing very rapidly. As 3D media, including augmented reality (AR), virtual reality (VR), and mixed reality (MR) devices and holograms, become more prevalent, data volumes are expected to increase even more rapidly. Specifically, the amount of data storage worldwide in 2040 is expected to reach 10²⁴ to 10²⁹ bits (Summary report, Technology Working Group Meeting on future DNA synthesis technologies; Sep. 14, 2017, Arlington, VA).

Currently, the data storage medium for major devices such as computers and smartphones is flash memory, and flash memory has a data density of approximately 1 bit per 1 pg. To provide flash memory corresponding to the amount of data needed in 2040, 10¹⁴ kg of silicon wafers would be needed, but the supply of silicon wafers in 2040 is expected to be approximately 108 kg, which is far short of this demand. In addition, existing data storage media have a limitation that the data retention period is approximately 10 years.

Accordingly, attempts are continuously being made to develop new data storage media, one of which is a DNA storage device. Using DNA as a storage medium can overcome the data storage density, which is a shortcoming of existing storage media, and enables information to be stored stably even under physical shock. In addition, DNA can store 2×10²⁴ bits, corresponding to 10⁹ kg of flash memory, with 1 kg of synthesis, making it suitable for storing large amounts of data, and particularly, it has the advantage of having a very long storage period for stored data. However, errors occurring in DNA synthesis cause a decrease in data storage density and information loss, which is an obstacle to the commercialization of DNA storage devices.

Particularly, in photochemical DNA synthesis methods, the protective groups at the ends of DNA are deprotected by lighting, and therefore light reaches an undesired region through diffraction, scattering, and reflection. In addition, there is a problem that there are regions where sufficient light does not reach where DNA is to be synthesized. These problems lead to deprotection of DNA at non-targeted sites, and failure to deprotect DNA at targeted sites, resulting in errors in DNA synthesis.

### DISCLOSURE

### Technical Problem

In light of the above situation, the present inventors studied a method of evenly applying light to a site where DNA is to be synthesized in a photochemical DNA synthesis process, and invented a method of deprotecting and capping DNA first at the region excluding an undesired DNA synthesis region, and deprotecting and coupling DNA in the desired DNA synthesis region.

Using the above method, it is possible to overcome the phenomenon in which, in the step of deprotecting the protective groups at the ends of DNA by lighting, the protecting groups are not guaranteed to be deprotected because the light does not provide sufficient light to the edges of the DNA synthesis region.

Therefore, the present invention is directed to providing a DNA synthesis method using the above-described principle.

### Technical Solution

One aspect of the present invention provides a DNA synthesis method, which includes:
(a) deprotecting DNA located in a reverse-irradiation region by applying light on the reverse-irradiation region of a DNA synthesis substrate;
(b) capping the ends of DNA located in the reverse-irradiation region by providing a capping material to the DNA synthesis substrate;
(c) deprotecting DNA located in the irradiation region by applying light to the irradiation region of the DNA synthesis substrate; and
(d) providing a nucleotide solution to be synthesized,
wherein edges of the reverse-irradiation region and the irradiation region overlap.

Another aspect of the present invent ion provides a DNA synthesis method, which includes:
(a) providing a material for selecting a DNA synthesis site to a DNA synthesis substrate;
(b) deprotecting DNA located in a reverse-irradiation region by applying light to the reverse-irradiation region of the DNA synthesis substrate;
(c) capping the ends of DNA located in the reverse-irradiation region by providing a capping material to the DNA synthesis substrate;
(d) reproviding the material for selecting a DNA synthesis site to the DNA synthesis substrate;
(e) deprotecting DNA located in an irradiation region by applying light to the irradiation region of the DNA synthesis substrate; and
(f) providing a nucleotide solution to be synthesized,
wherein edges of the reverse-irradiation region and the irradiation region overlap.

### Advantageous Effects

Using a DNA synthesis method according to an embodiment of the present invention, errors caused by insufficient light arrival during synthesis can be significantly reduced.

### DESCRIPTION OF DRAWINGS

FIG. 1 schematically shows the cause of synthesis errors generated in photochemical DNA synthesis.

FIG. 2 shows the result of measuring the amount of light applied to the synthesis region of the DNA synthesis substrate in a photochemical DNA synthesis process.

FIG. 3 shows an example of the process of deprotecting the ends of DNA and adding a new nucleotide in a photochemical DNA synthesis method.

FIG. 4 shows an example of the process of deprotecting the ends of DNA by a deprotecting molecule supplier in photochemical DNA synthesis and adding a new nucleotide.

FIG. 5A shows the process of deprotecting DNA present in a reverse-irradiation region by first applying light to the region (reverse-irradiation region) excluding a DNA synthesis region (irradiation region) on a DNA synthesis substrate.

FIG. 5B shows the process of capping the ends of deprotected DNA present in a reverse-irradiation region by providing a capping material to a DNA synthesis substrate.

FIG. 5C shows the process of deprotecting DNA present in an irradiation region by applying light to a DNA synthesis region (irradiation region).

FIG. 6A shows one type of a DNA synthesis substrate.

FIGS. 6B and 6C show examples of applying light by moving a reverse-irradiation region of a DNA synthesis substrate in the horizontal direction when light is applied to the reverse-irradiation region.

FIG. 6D shows an effective DNA synthesis region (irradiation region).

FIG. 6E shows an example of a DNA synthesis region (irradiation region) to which light is applied.

### MODES OF THE INVENTION

Hereinafter, embodiments of the present invention will be described in detail with reference to the accompanying drawings so that those of ordinary skill in the art can easily carry out the present invention. However, in the description of the present invention, when detailed description of the related known technology or configurations is determined to unnecessarily obscure the subject matter of the present invention, the detailed description will be omitted. In addition, parts that have similar functions and actions are indicated by the same symbols throughout the drawings. Meanwhile, throughout the specification, when one part “includes” a component, it means that it may also include other components, not excluding components unless particularly stated otherwise.

Photochemical DNA synthesis methods use a method of selectively deprotecting protective groups attached to the ends of a newly added nucleotide by applying light.

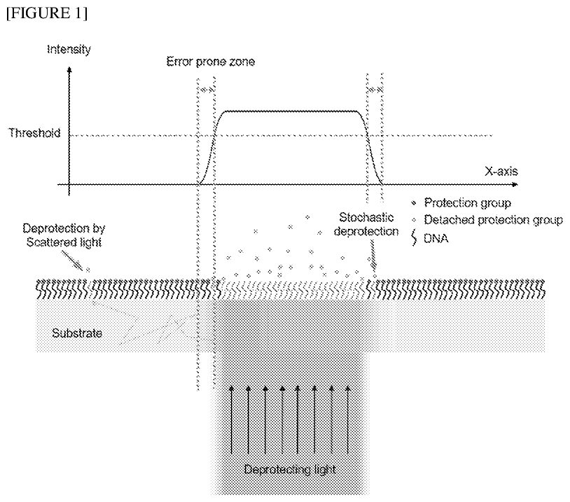

FIG. 1 schematically shows the cause of synthesis errors generated in photochemical DNA synthesis.

When light (UV or 100 to 500 nm wavelength) is selectively applied to a synthesis region of a DNA synthesis substrate using a photomask or a digital micromirror device (DMD), the light may be reflected, scattered, or spread by diffraction from the DNA synthesis substrate, and the light may reach an unnecessary region. Light reaching an unnecessary region deprotects the protective groups at the ends of DNA in the corresponding region, which leads to errors of DNA synthesis.

A more important problem is that there are regions of the DNA synthesis substrate where sufficient light does not reach a target location, and in these regions, stochastic deprotection of the protecting groups occurs.

The corresponding region is marked as an error-prone zone in FIG. 1. Some protecting groups are deprotected since light reaching the error-prone zone does not reach the threshold required for deprotection of the protecting groups, but some remain intact without being removed.

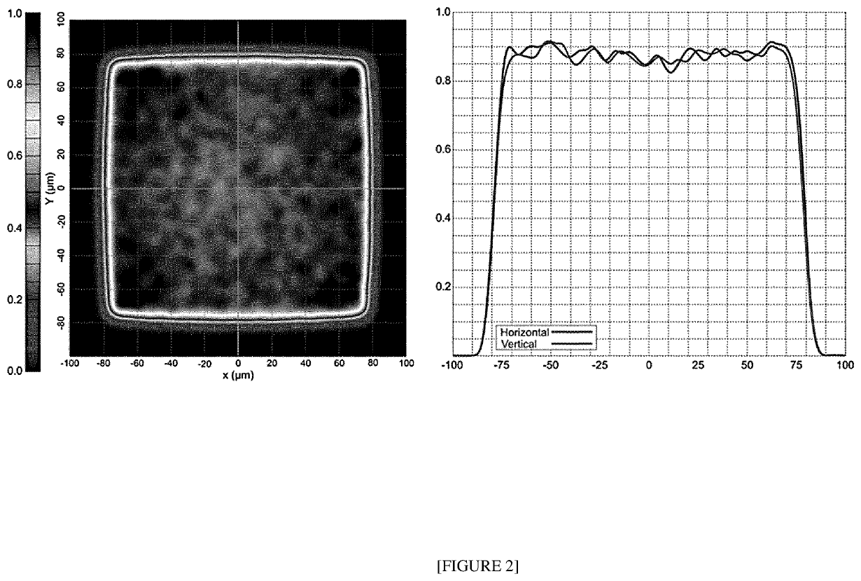

It can also be confirmed in FIG. 2 that there is a region where insufficient light reaches the target location of the DNA synthesis substrate.

FIG. 2 shows the result of measuring the amount of light applied to the synthesis region of the DNA synthesis substrate in a photochemical DNA synthesis process.

The left picture in FIG. 2 is a top view of light irradiation to a DNA synthesis site, and the intensity of light at each position is expressed in color. The right picture shows the intensity of light drawn on the x-axis (blue) and the y-axis (green). When light is applied to the DNA synthesis site, the central area maintains a constant amount of light, but the amount of light reaching the edges is reduced with a gradient. That is, the contrast ratio of the light pattern projected onto the substrate is not perfect.

As described in the description of FIG. 1, the edges (corresponding to the error-prone region in FIG. 1) gradually decrease in light intensity, and therefore cannot provide sufficient light for deprotection, and as a result, deprotection may occur at certain locations, but not at other locations. Since the probabilistic deprotection is very random, in each cycle of DNA synthesis, even if the DNA strand is not deprotected in the (n−1)^{th} DNA synthesis, it may be deprotected in the (n)^{th} DNA synthesis, and when a new nucleotide is added (coupling), a deletion error may occur in the (n−1)^{th} sequence.

That is, whether deprotection occurs in the (n)^{th} DNA synthesis is independent of whether deprotection occurs in the previous (n−1)^{th} DNA synthesis. Because of deletion errors in such edges (error-prone regions), capping is required to block the ends of the DNA or a nucleic acid being synthesized to prevent further synthesis in each synthesis cycle, and after synthesis is complete, a process is required to remove DNA that is shorter than the target length. The errors occurring at the edges become more significant as the length to surface ratio increases, i.e., the size of the light-applied area decreases, making it difficult to reduce the size of a single spot to increase the number of possible simultaneous syntheses in microarray DNA synthesis.

To solve the problems described in FIGS. 1 and 2, the present inventors invented a method of deprotecting and capping DNA first in the region excluding a desired DNA synthesis region, and then deprotecting and coupling the DNA in the desired DNA synthesis region.

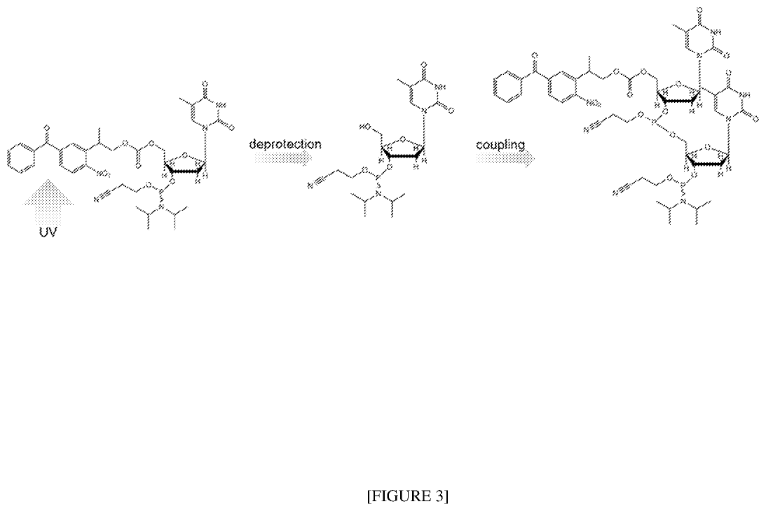

First, FIG. 3 shows an example of the process of deprotecting the ends of DNA and adding a new nucleotide in a photochemical DNA synthesis method.

When the deprotecting group at one end of the DNA is removed by light, the —OH group at the 5′-end of the DNA is exposed, allowing a new nucleotide to bind to the end of the DNA strand being synthesized. Since the nucleotide provided for synthesis has a protecting group binding to one end, additional DNA synthesis does not occur without a separate deprotection process.

Protecting groups that react directly with light and are detached include BzNPPOC, NPPOC, and SPh-NOOPC.

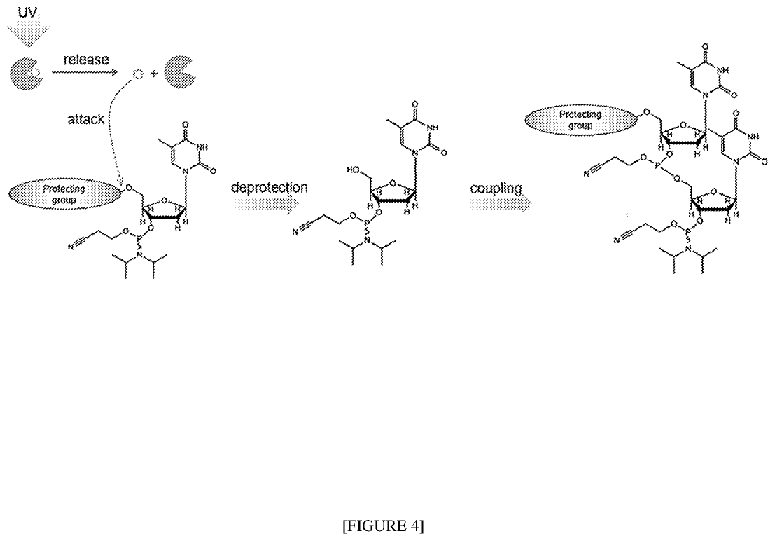

FIG. 4 shows an example of the process of deprotecting the ends of DNA by a deprotecting molecule supplier in photochemical DNA synthesis and adding a new nucleotide.

When exposed to light, a deprotecting molecule supplier receives the light and releases an active deprotecting molecule, which attacks and detaches the protecting group. As a result, the —OH group is exposed at the 5′-end of the DNA, allowing the new nucleotide to bind to the end of the DNA strand being synthesized.

As the deprotecting molecule supplier, hydroquinone may be used, as the active deprotecting molecule, a hydrogen ion (H⁺) may be used, and as the protecting group, DMT may be used.

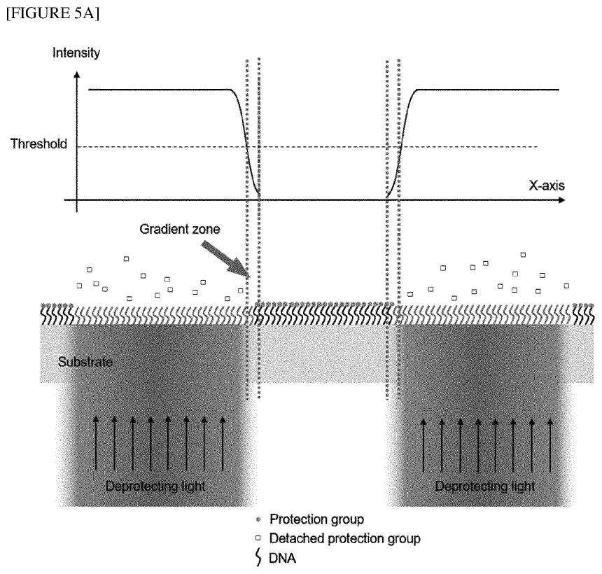

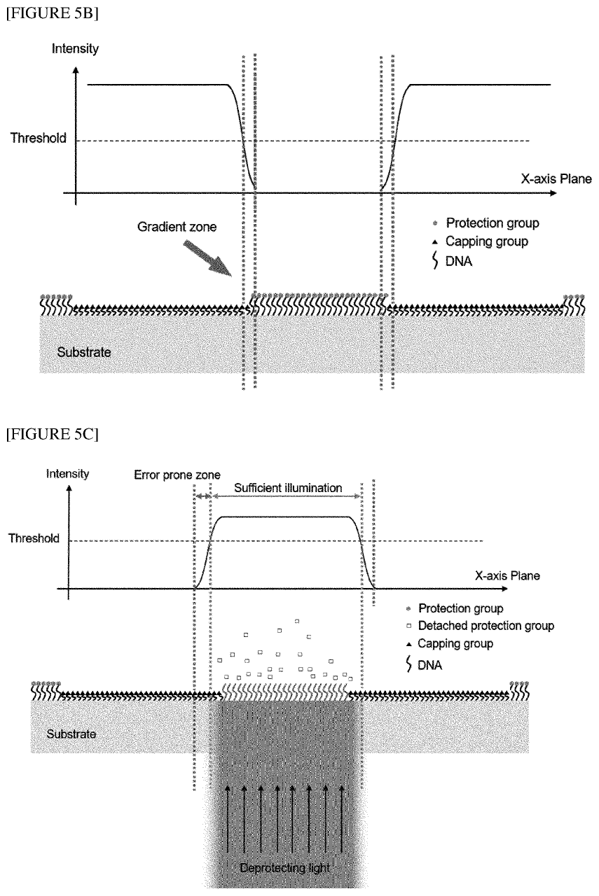

FIG. 5 shows a DNA synthesis method according to one example of the present invention step by step, and the method includes the following steps:
(a) deprotecting DNA located in a reverse-irradiation region by applying light to the reverse-irradiation region of a DNA synthesis section;
(b) capping the end of the DNA located in the reverse-irradiation region by providing a capping material to the DNA synthesis section;
(c) deprotecting DNA located in an irradiation region by applying light to the irradiation region of the DNA synthesis section; and
(d) providing a nucleotide solution to be synthesized, wherein edges of the reverse-irradiation region and the irradiation region overlap.

The term “reverse-irradiation region” used herein refers to the region excluding the region where DNA is to be synthesized on a DNA synthesis substrate, and “irradiation region” refers to the region where DNA is to be synthesized on a DNA synthesis substrate.

FIG. 5A corresponds to step (a) of the above method, and shows the process of applying light to the region (reverse-irradiation region) excluding the DNA synthesis region (irradiation region) on the DNA synthesis substrate to deprotect DNA present in the reverse irradiation area. In FIG. 5A, the region where deprotecting light is applied is the reverse-irradiation region, and deprotection occurs in the DNA located in the reverse-irradiation region by light.

The insufficient light arrival phenomenon as described in FIGS. 1 and 2 also occurs when light is applied to the reverse-irradiation region, and a gradient zone where insufficient light reaches the edges of the reverse-irradiation region also exists.

In FIG. 5A, DNA oligos are illustrated as if they were already present in the entire region of the DNA synthesis substrate, but the present invention is not limited thereto. For example, this step may be performed in the state where the 1-mer DNA protected by protecting groups binds to the DNA synthesis substrate, which is the first step of DNA synthesis, or a functional group capable of being coupled with DNA binds to the DNA synthesis substrate, and this step may also be performed while 1-mer DNA protected by a protecting group is linked to the functional group.

FIG. 5B corresponds to the step (b) of the method and illustrates the process of capping a deprotected end of the DNA present in the reverse-irradiation region by providing a capping material to the DNA synthesis substrate.

A capping material that can be used is acetic anhydride or N-methylimidazole, and has the property of not being detached from DNA by light.

As described in FIG. 5A, there is also a gradient zone in the reverse-irradiation region where insufficient light reaches, and in this region, some DNAs may be capped, but some may still have deprotecting groups.

Through the steps of FIGS. 5A and 5B, the effective area where DNA synthesis can occur is reduced.

FIG. 5C shows the process of deprotecting DNA present in an irradiation region by applying light to a DNA synthesis region (irradiation region) after capping the reverse-irradiation region.

When applying light to the irradiation region, it is preferable to apply light over a wider area than the irradiation region to ensure that the light reaches the entire irradiation region evenly. That is, edges of the reverse-irradiation region and the irradiation region overlap, and may be 1 nm to 100 μm apart.

When light is applied wider than the irradiation region, the error-prone region described in FIG. 1 overlaps with the gradient zone described in FIG. 5A, and this overlapping part is not affected by light because the ends of DNA are blocked by the capping material. Therefore, DNA deprotection and coupling occur only in the regions where light reaches evenly. Light irradiation to the reverse-irradiation region and the irradiation region may be performed for, but is not limited to, 0.1 nanoseconds to 10 minutes.

Meanwhile, in FIG. 5, an example is given of a case where the 5′-end of DNA is directly detached by light and protected with a photo-labile protecting group, but the present invention is not limited thereto. For example, as shown in FIG. 2, the present invention can also be applied to cases where light reacts with a deprotecting molecule supplier to release an active deprotecting molecule, and the active deprotecting molecule removes a protecting group such as DMT attached to the 5′-end of DNA.

When this deprotecting molecule supplier is used, the DNA synthesis method may include:
(a) providing a material for selecting a DNA synthesis site to a DNA synthesis section;
(b) deprotecting DNA located in a reverse-irradiation region by applying light to the reverse-irradiation region of the DNA synthesis section;
(c) capping the ends of DNA located in the reverse-irradiation region by providing a capping material to the DNA synthesis section;
(d) deprotecting DNA located in an irradiation region by applying light to the irradiation region of the DNA synthesis section; and
(e) providing a nucleotide solution to be synthesized,
wherein edges of the reverse-irradiation region and the irradiation region overlap.

The term “material for selecting a DNA synthesis site” used herein refers to a material required to synthesize DNA only at a desired site in DNA synthesis, and a deprotecting molecule supplier such as a hydrogen ion-generating material, a hydroxide ion-generating material, or a divalent cation-donating material.

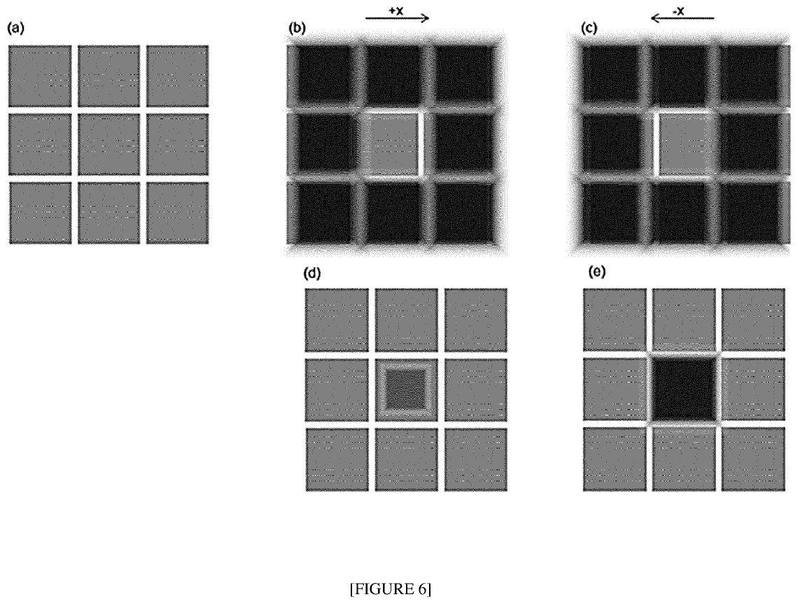

FIG. 6A shows one type of a DNA synthesis substrate.

FIGS. 6B and 6C show examples of applying light by moving the substrate in the horizontal direction when light is applied to a reverse-irradiation region of a DNA synthesis substrate to sufficiently reduce the effective area for DNA synthesis. The central square is an irradiation region where DNA is to be synthesized, and the remaining square is a reverse-irradiation region.

FIG. 6B shows the application of light to the DNA synthesis substrate by moving the reverse-irradiation region in the +x direction with respect to the DNA synthesis substrate, and FIG. 6C shows the application of light to the DNA synthesis substrate by moving the reverse-irradiation region in the −x direction with respect to the DNA synthesis substrate. The light application may also be performed in the same manner in the +y and −y directions.

FIG. 6D shows the effective DNA synthesis region (irradiation region) when light is applied after moving the reverse-irradiation region in the +x, −x, +y, and −y directions with respect to the DNA synthesis substrate. It can be seen that the effective area is reduced compared to the original square size.

FIG. 6E shows an example of the application of light to the DNA synthesis region (irradiation region), in which the light irradiation range is wider than the effective DNA synthesis region of FIG. 6D, but actual DNA synthesis occurs only in the effective range.

The DNA synthesis method of the present invention described above may be used to selectively deprotect a protecting group such as DMT or BzNPPOC with high resolution and introduce a desired monomer into a desired part in the form of a microarray, but the present invention is not limited thereto.

All of the DNA synthesis processes described in FIGS. 1 to 5 are DNA syntheses mediated by phosphoramidite using an organic solvent, but the present invention may also be applied to a DNA polymerase-based method.

Among DNA synthases, unusually, terminal deoxynucleotidyl transferase (TdT) is capable of synthesizing single-stranded DNA of an arbitrary sequence without template DNA. The most important issue in DNA synthesis using TdT is how to introduce a desired monomer among A, G, T, and C, which may be solved using the present invention.

By irradiating the reverse-irradiation region with light to release divalent cations from the material for selecting a DNA synthesis site and introducing a capped DNA monomer to allow them to be coupled by TdT activated by the divalent cations, no more DNA synthesis can occur in this region.

Alternatively, by applying light to the reverse-irradiation region when a DNA synthesis substrate is coated with protecting groups, a deprotecting molecule supplier in the solution can allow an active deprotecting molecule (hydrogen ion) to release and to detach the protecting groups. After removal of the protecting groups, a capped DNA monomer may be introduced to allow coupling mediated by activated TdT, thereby preventing further DNA synthesis in this region.

1. A DNA synthesis method, comprising:
(a) deprotecting DNA located in a reverse-irradiation region by applying light on the reverse-irradiation region of a DNA synthesis substrate;
(b) capping the ends of DNA located in the reverse-irradiation region by providing a capping material to the DNA synthesis substrate;
(c) deprotecting DNA located in the irradiation region by applying light to the irradiation region of the DNA synthesis substrate; and
(d) providing a nucleotide solution to be synthesized,
wherein edges of the reverse-irradiation region and the irradiation region overlap.

2. The method of claim 1, wherein, in (a) and (c), light is applied for 0.1 nanoseconds to 10 minutes.

3. A DNA synthesis method, comprising:
(a) providing a material for selecting a DNA synthesis site to a DNA synthesis substrate;
(b) deprotecting DNA located in a reverse-irradiation region by applying light to the reverse-irradiation region of the DNA synthesis substrate;
(c) capping the ends of DNA located in the reverse-irradiation region by providing a capping material to the DNA synthesis substrate;
(d) reproviding the material for selecting a DNA synthesis site to the DNA synthesis substrate;
(e) deprotecting DNA located in an irradiation region by applying light to the irradiation region of the DNA synthesis substrate; and
(f) providing a nucleotide solution to be synthesized,
wherein edges of the reverse-irradiation region and the irradiation region overlap.

4. The method of claim 3, wherein, in (b) and (e), light is applied for 0.1 nanoseconds to 10 minutes.

5. The method of claim 3, wherein, in (a) and (d), the material for selecting a DNA synthesis site is a hydrogen ion-generating material, a hydroxide ion-generating material, or a divalent cation-donating material.

6. The method of claim 5, wherein the hydrogen ion-generating material is hydroquinone.

7. The method of claim 5, wherein the hydroxide ion-generating material is quinone.

8. The method of claim 5, wherein the divalent cation-donating material is DMNP-EDTA.

9. The method of claim 1, further comprising washing the DNA synthesis substrate after providing a nucleotide solution.

10. The method of claim 1, wherein the applying of light to the reverse-irradiation region of the DNA synthesis substrate is i) applying light to match the reverse-irradiation region, or ii) applying light to a region moved horizontally or vertically from the reverse-irradiation region.

11. The method of claim 1, wherein the capping material is acetic anhydride or N-methylimidazole.

12. The method of claim 3, further comprising washing the DNA synthesis substrate after providing a nucleotide solution.

13. The method of claim 3, wherein the applying of light to the reverse-irradiation region of the DNA synthesis substrate is i) applying light to match the reverse-irradiation region, or ii) applying light to a region moved horizontally or vertically from the reverse-irradiation region.

14. The method of claim 3, wherein the capping material is acetic anhydride or N-methylimidazole.
